(19) United States
(12) Patent Application Publication
(10) Pub. No.: US 2023/0218369 A1
(43) Pub. Date: Jul. 13, 2023

(54) SYSTEMS, DEVICES, AND METHODS FOR PROVIDING INFLATABLE ISOLATION AND NEGATIVE ENVIRONMENT FIELD

(71) Applicant: Kelly LUCAS DDS, Wasilla, AK (US)

(72) Inventor: Kelly Lucas, Wasilla, AK (US)

(21) Appl. No.: 18/000,828

(22) PCT Filed: Jun. 8, 2021
(86) PCT No.: PCT/US2021/036467

§ 371 Date: Dec. 6, 2022

Related U.S. Application Data
(60) Provisional application No. 63/035,945, filed on Jun. 8, 2020.

(51) Int. Cl. A61C 5/82; A61C 17/08; A61M 16/04

(52) U.S. Cl. CPC A61C 5/82; A61C 17/08; A61M 16/0493

(57) ABSTRACT

An intraoral inflatable isolator (III) for an oral cavity of a patient, the III including: a compressed-air port; and an inflatable membrane dimensioned to be insertable into the mouth and connected to the compressed-airport, the membrane being configured to inflate upon application of compressed air from the compressed-air port to isolate within the oral cavity.

### CROSS REFERENCE TO RELATED APPLICATIONS

This application claims priority to U.S. Provisional patent application Ser. No. 63/035,945, filed Jun. 8, 2020, the entire disclosure of which is hereby expressly incorporated by reference herein.

### FIELD

The present application relates to an integrated intraoral, extraoral, and external isolation and negative environment system and methods for using the same, and more specifically, to an inflatable intraoral and extraoral isolation and negative environment system and methods for using the same.

### BACKGROUND

Intraoral isolation devices assist oral health professionals by opening a patient's mouth, vacuuming saliva and debris from the patient's mouth, retracting the patient's soft tissue, lighting the patient's mouth, and/or protecting the patient's throat and airway. While many different types of related art intraoral isolation devices currently perform one or more of these functions (for example, the related art depicted in FIG. 23), they may be limited due to being constructed of rigid or semi-rigid materials or require invasive retention clamps. The related art has a number of deficiencies discussed below.

First, for example, the rigid or semi-rigid construction of related art intraoral isolation devices may cause discomfort for the patient during and after installation. In many cases the mouth opening is smaller than the oral cavity, so intraoral isolation devices must be small enough to fit through the patient's mouth opening (lips) but large enough to provide all the support, functionality, and isolation coverage of the oral cavity. Due to their rigid size and shape, related art intraoral isolation devices are difficult to place in the patient's mouth and render the patient uncomfortable during installation (and often throughout the duration of the procedure). Further related art intraoral isolation devices such as a rubber dam require a clamp to be placed on a tooth to anchor the rubber dam, this requires anesthesia and there may be damage to the gingiva associated with the clamp.

Second, related art intraoral isolation devices may provide limited vacuuming during oral health procedures. Patients constantly produce saliva during oral health procedures which may negatively impact the procedure. Oral health professionals use intraoral isolation devices to block saliva from areas specific to the procedure and separate vacuums to remove saliva and debris that accumulate in the patient's mouth. However, these vacuums must be manually maneuvered and operated by an oral health professional to reach different areas of the oral cavity. Further, while some related art intraoral isolation devices have integrated vacuum, the rigid or semi-rigid shape of related art intraoral isolation devices limits their ability to reach certain areas of the oral cavity, such as the parotid duct.

Third, the rigid structure of related art intraoral isolation devices do not easily adjust to the changing size of opening and retraction requirements of a patient's mouth through the course of a procedure. Soft tissues in a patient's mouth (including the tongue and cheeks) must therefore collapse around hard tissues (bone and teeth) on which oral health professionals work. The workspace is restricted, limiting access and increasing the risk of inadvertent soft tissue injury. While some related art intraoral isolation devices provide limited adjustments, the rigid or semi-rigid structures have predetermine size and shape. Thus, as soft tissue retraction and protection needs change throughout the procedure, the oral health professionals can attempt to manually adjust the intraoral isolation device(s) as needed or must replace the device with one of a different size and/or shape. As described above, this process is difficult for the oral health professional and uncomfortable for the patient.

Fourth, related art intraoral isolation devices are not configured for use by a plurality of patients whose mouth openings and oral cavities vary in size and shape. As related art intraoral isolation devices have rigid or semi-rigid structures that determine their size and shape, an oral health professional must keep available multiple intraoral isolation devices of various sizes and shapes on hand to accommodate a plurality of patients. Keeping an inventory of extra intraoral isolation devices on hand can be expensive, and determining the correct-sized device for a particular patient can be a time-consuming process. Additionally, if the first device that is installed in a patient turns out to be the wrong size, removing the first device and installing a second device causes the patient additional and unnecessary discomfort.

Fifth, related art intraoral isolation devices may provide limited lighting during oral health procedures. The oral cavity is inherently dark, so oral health professionals require light to illuminate their work area. Solutions that address this problem include intraoral isolation devices that deliver light through a cable, external overhead lights, and hands-free lights strapped to the oral health professional's head. External lights have a limited ability to deliver light into all areas of the mouth, particularly while the oral health professional is working. Lights included with related art intraoral isolation devices are limited by the device's rigid or semi-rigid structure, adequately illuminate certain parts of the oral cavity, and are not readily adjustable.

Sixth, related art intraoral isolation devices may provide limited airway protection with comfortable breathability during oral health procedures. For example, devices such as a rubber dam may protect the throat, but may create psychologically or physically breathing difficulty by requiring breathing around this barrier. Further, due to the rigidity of many related art intraoral isolation devices and the unique contours of an individual patient's mouth, complete throat protection while comfortably maintaining breathability is difficult to achieve.

Seven, related art intraoral isolation devices provide, at best, limited noise suppression during oral health procedures. Patients undergoing oral health procedures are often subject to loud noises from oral health professionals' tools (e.g., drills). During such oral health procedures, loud noises inside the oral cavity occur close to the patient's ears and can be offensive. Related art devices have very limited noise reduction property providing by an additional barrier between the patient's oral cavity and ears. However, due to their rigidity and the unique contours of individual patients' mouths, related art intraoral isolation devices are unable to provide sufficient noise reduction over an effective surface area in the oral cavity.

Eight, current intraoral isolation systems do not provide integrated attachment points or have integrated electronic and/or communication pathways for adjunct use such as micro intraoral cameras and/or other sensors.

Meanwhile, the use of various dental instruments, such as high speed drills and air/water syringes, along with other procedures may produce splatter micro-droplets, and/or aerosols which can contain microorganisms and other potential contaminants that may be a hazard for other patients and dental personnel. Related art vacuum devices (for example, FIG. 24) can assist oral health professionals by ameliorating splatter, micro-droplets, and/or aerosols that may be produced upon delivery of dental care. However, these related art extraoral vacuum devices are standalone units that, when deployed, are designed to be placed immediately in front of a patient's mouth. Accordingly, these devices may interfere with light and visibility and access into the oral cavity during a dental procedure. Further, these units are large and noisy and may interfere with the dental workers and the ergonomics of a dental operatory. Additionally, because the vacuum is separated from a patient's mouth, the likelihood of escaped aerosols is significant. Moreover, because the related art extraoral vacuum devices are located remotely from the patient, even if the related art devices are sufficiently capable of ameliorating microdroplets/aerosol, this effectiveness is reduced anytime a patient moves. Thus an operator or assistant may have to reposition the extraoral vacuum many times during a procedure.

Ninth, there are numerous applications outside of the dental space that could benefit from negative environments. For example, surgical sites on a body could benefit from an external vacuum negative environment, as this may reduce debris, aerosol, splatter, micro-splatter, droplets, micro-droplets or any other infective, as well as noxious, caustic, or poisonous gaseous or any other material caused by the clinical situation or surgical procedure.

Accordingly, there is a need for improved intraoral, extraoral, and external isolation and negative environment systems, and embodiments of the present disclosure are directed to this and other considerations.

### SUMMARY

Briefly described, embodiments of the present disclosure can comprise an inflatable intraoral isolation device (III). The III may have an integrated vacuum and negative environment system, creating an Inflatable Isolation Negative Environment System (IINES). The inflatable intraoral isolation (III) may have an inflatable flexible membrane containing one or two separately inflatable and detachable bite blocks, separately inflatable vacuum chambers, pockets and tubes, integrated inflatable support structures, channels with holes for the removal of saliva and debris, an umbilical or a plurality of umbilicals, and/or a breathing channel

The breathing channel may include separate pathways for inhalation of air, inhalation of therapeutic gases, exhalation of air, and exhalation of therapeutic gases, or the pathways may be combined. The umbilical may provide pathways for compressed air, vacuum, various types of light, and electrical, data, and communication pathways for adjuncts.

The flexible inflatable membrane may also include one or more connectors for subsystems distribution and pathways for vacuum, light for visibility of the mouth and teeth for work, light for disinfection, light for materials curing, water, compressed air, electricity, and/or two way data and communication pathways. Connectors could also include an integrated air/water port, an integrated port for medicaments for disinfection such as low dose hydrogen peroxide, and attachment points for adjuncts such as micro intraoral cameras, sensors (moisture, microbial, sound, chemical, audio/visual), microprocessors, sound emitters (such as negative sound wave technology and content such as music), and integrated pathways for electronics support and communications for these adjuncts.

The membrane may have attached or integrated one or more bite block(s) and/or inflatable structural systems such as inflatable rib retraction systems. The membrane may have integrated inflatable areas, chambers, tubes or pockets. The intraoral membrane may include vacuum ports for saliva, liquid and debris removal, and vacuum pistons for manipulation of the shape of the membrane (e.g., tongue retraction by means of pulling toward the bite block by retraction of the tubes upon vacuum application). Shape manipulation by inflation or vacuum pistons may be calibrated for different shapes and sizes by amount of vacuum and/or compressed air applied. Light for visibility, disinfection, and materials curing may be provided or diffused by the membrane itself or through channels within the membrane. The inflatable membrane may be flavored. The membrane may contain systems to spray water in a certain area for constant or intermittent debris removal, cooling, and visibility. The membrane may contain pathways for liquids or gels close to certain structures or anatomy for indirect cooling or heating, distribution of medicaments, materials curing or manipulation, and other functions.

The integrated external vacuum negative environment system (EVNES) (which may be integrated with the III or be a standalone unit) may receive light for visibility, light for disinfection, compressed air, and vacuum through similar (or the same) pathways, connectors, and umbilical systems as the intraoral isolator aspect (III). The EVNES may be an extraoral vacuum negative environment system, but this is merely an example.

The EVNES is made of a flexible membrane with inflatable ribs, inflatable pockets or chambers, and/or inflatable support structures that can be of various designs for different structural characteristics when inflated, partially inflated, or deflated. The EVNES may have integrated vacuum pistons, pockets, and chambers for shape manipulation, vacuum tubes and ports, and compressed air tubes and ports to allow complex dynamic air architectures just outside a patients' mouth to effectively manage splatter, microdroplets, and aerosols created by dental procedures. The EVNES may also include rigid and/or semi-rigid members or structures to maintain its shape.

The membrane may have an accordion type construction to allow easy collapsibility against the force of an operator or assistant's a hand(s), finger(s) or instrument(s) when placed into or around the patients' mouth. Thus, the EVNES may be comfortable for operators and the patient, and continue to provide splatter, microdroplet and aerosol reduction or elimination through uncollapsed or cut-out portions. The shape of the extraoral vacuum negative environment system (EVNES) aspect may resemble a lampshade or a hovercraft skirt. The EVNES may be circular, conical, or elliptical (e.g., mimicking a shape of the patients' mouth) or be of various other shapes that may enhance air dynamics for effective extraoral vacuum and negative environment production. The EVNES may be of various heights and/or variable heights. The EVNES may be constructed in a single layer or several layers and may be of varying thicknesses.

A drape may be provided at an outer edge of the III and/or an inner edge of the extraoral aspect (EVNES) to cover the patients' face, head, neck, and/or upper body.

In certain embodiments the inflatable intraoral isolator (III) may be provided without a drape or an EVNES.

In another aspect, a method for using an III is disclosed. The method may include inserting the deflated intraoral isolation isolator (III) into the patients' mouth and oral cavity. This may include inserting a complement of inputs and outputs appropriate for a procedure such as a compressed air line into the connector or umbilical. If included, the drape and/or EVNES will be left outside or substantially outside the mouth. The III can be inflated. Inflating may include inflating one or more bite blocks as well as certain individual chambers with compressed air to facilitate completion of the oral health procedure. Inflation may also include inflating pockets or chambers formed by the membranes that can provide variable structural support and/or sound manipulation.

Inflating may include inflating the EVNES, e.g., inflating inflatable ribs, pockets, and/or chambers of the EVNES for provision of support for the extraoral vacuum ports. In some cases, the EVNES may include rigid or semi-rigid members or structures that do not require inflation. These rigid and/or semi-rigid structures may support of the physical shape of the EVNES and prevent collapse of the vacuum system of the EVNES. The rigid and/or semi-rigid structures can be in addition to or in lieu of inflatable support structures.

The inflatable bite blocks, rib systems, pockets, and chambers of the III and/or EVNES may be adjusted as necessary to accommodate the evolving needs of the oral health procedure by adding or removing compressed air. The vacuum pistons, chambers or pockets may be activated with vacuum to manipulate the shapes of the respective subsets of the TINES. The method may also include inserting an input into the connector to vacuum saliva and debris from the patient's oral cavity. Vacuum supplied to the unit may also provide vacuum for creating the extraoral vacuum and negative environment system of an optional EVNES.

The method may include inserting an input to a connector or several connectors to deliver light for visibility, disinfection, and/or materials curing to the patient's oral cavity and light for visibility and/or disinfection to the extraoral vacuum negative environment system. Further, electronic, data, and communication leads may be transited through an umbilical and/or attached to connectors for use by both the intraoral and extraoral aspects of the system. Once the oral health procedure, or specific portion thereof, requiring the III and/or EVNES is complete, the bite block(s) and inflatable chambers may be deflated to facilitate easy removal of the entire system from the patient's mouth. An emergency deflation port may be included in the system for immediate deflation and rapid emergency removal of the III and/or EVNES.

Further features of the disclosed design, and the advantages offered thereby, are explained in greater detail hereinafter with reference to specific embodiments illustrated in the accompanying drawings, wherein like elements are indicated by like reference designators.

### BRIEF DESCRIPTION OF THE DRAWINGS

FIGS. 1A and 1B illustrate an inflatable intraoral isolation device in deflated and inflated states according to some embodiments.

FIGS. 2A and 2B illustrate an inflatable intraoral isolation device in deflated and inflated states inside a patients mouth according to some embodiments.

FIGS. 3A and 3B illustrate a bite block with inflatable stops according to some embodiments.

FIG. 4 is an view of an inflatable intraoral isolation device with a bite block according to some embodiments.

FIG. 5 illustrates example port layouts in accordance with some embodiments.

FIGS. 6A and 6B illustrate example tongue retractor in accordance with some embodiments.

FIG. 7 illustrates example wall structures in accordance with some embodiments.

FIG. 8 illustrates example cheek retraction in accordance with some embodiments.

FIG. 9 illustrates a an internal membrane structure according to some embodiments.

FIG. 10 illustrates an III with a drape in accordance with some embodiments.

FIG. 11 illustrates an integrated vacuum in accordance with some embodiments.

FIGS. 12-14 illustrate example extraoral vacuum negative environment systems state in accordance with some embodiments.

FIG. 15 illustrates a negative environment created by a extraoral vacuum negative environment system in accordance with some embodiments.

FIGS. 16A and 16B illustrate example wall structures of a extraoral vacuum negative environment system according to some embodiments.

FIG. 17 illustrates an integrated III an EVNES according to some embodiments.

FIGS. 18A-19C illustrate an EVNES according to some embodiments.

FIGS. 20-22 illustrate an example headrest negative environment systems according to some embodiments.

FIG. 23 illustrates an example cheek retractor in the related art.

FIG. 24 illustrates an example vacuum device in the related art.

FIG. 25 is a block diagram of an illustrative computer system architecture according to an example embodiment.

FIGS. 26A-C illustrates an EVNES according to some embodiments.

### DETAILED DESCRIPTION

To facilitate an understanding of the principles and features of the various embodiments of the invention, various illustrative embodiments are explained below. Although certain example embodiments are described below, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

To facilitate an understanding of the principles and features of this disclosure, various illustrative embodiments are explained below. In particular, various embodiments of this disclosure are described as smoking devices and articles, and methods for producing and using smoking devices. Some embodiments of the invention, however, may be applicable to other contexts, and embodiments employing these applications are contemplated.

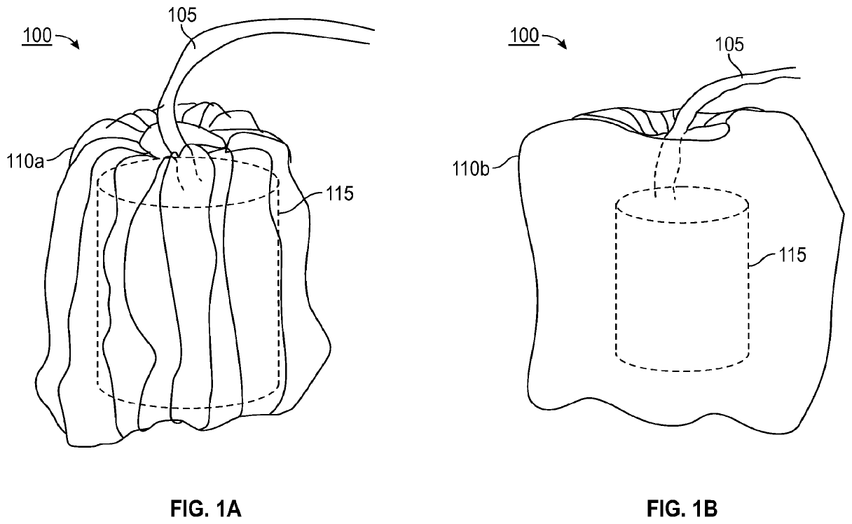

FIGS. 1A and 1B illustrate an inflatable intraoral isolation device (III) 100 according to some embodiments. III 100 includes an umbilical 105 that leads to a central port 115, surrounded by an inflatable membrane 110. The umbilical 105 may include, for examples, one or more pathways for compressed air, vacuum, various types of lights, electrical and data pathways, and/or a breathing channel (individually or collectively “inputs”). Although a single umbilical 105 is illustrated, one of ordinary skill will recognize that III 100 may include a plurality of umbilicals 105 attached port 115 and/or member 110.

Port 115 may serve as an attachment point for membrane 110 and umbilical 105. In some cases, port 115 may provide routing of the inputs from umbilical to guide compressed air, vacuum, light, and/or power to various channels of the membrane. Port 115 may include controls to selectively route inputs from umbilical 105 to chambers within member 110. For example, port 115 may actuate doors to open or close various channels of various inputs. To perform these actions, port 115 may include, for example, a processor (e.g., a microprocessor), electro-mechanical controls, and/or a transceiver to receive external instructions on which pathways to open and/or close.

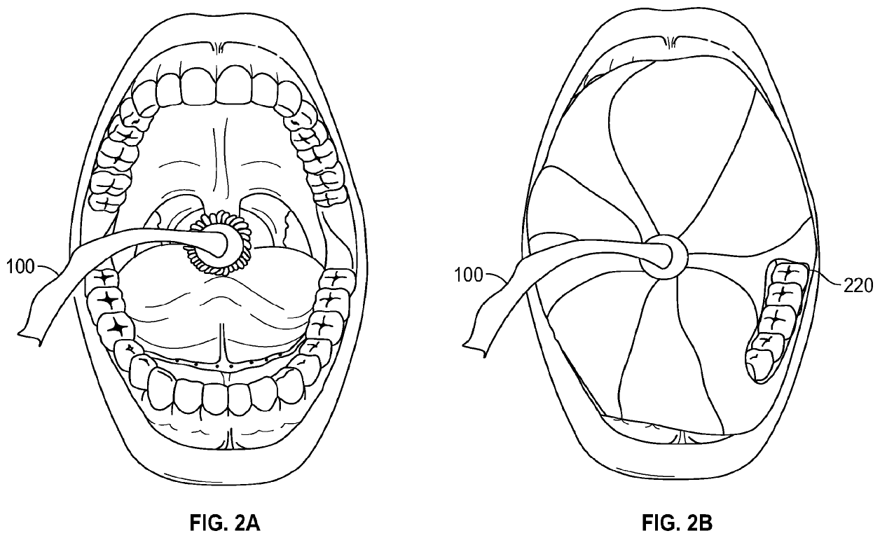

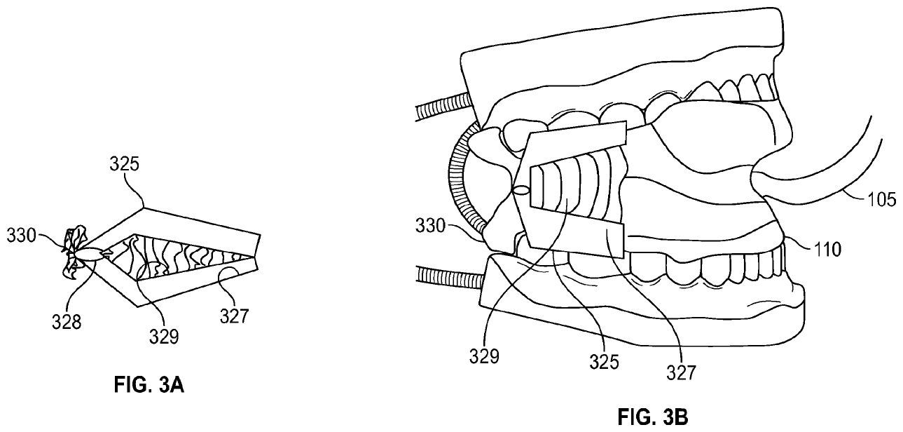

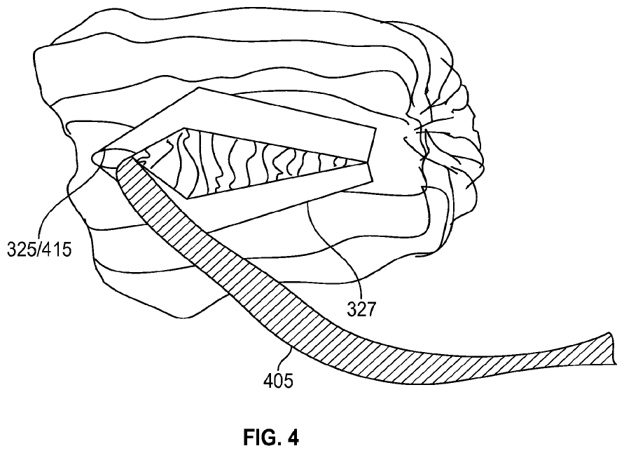

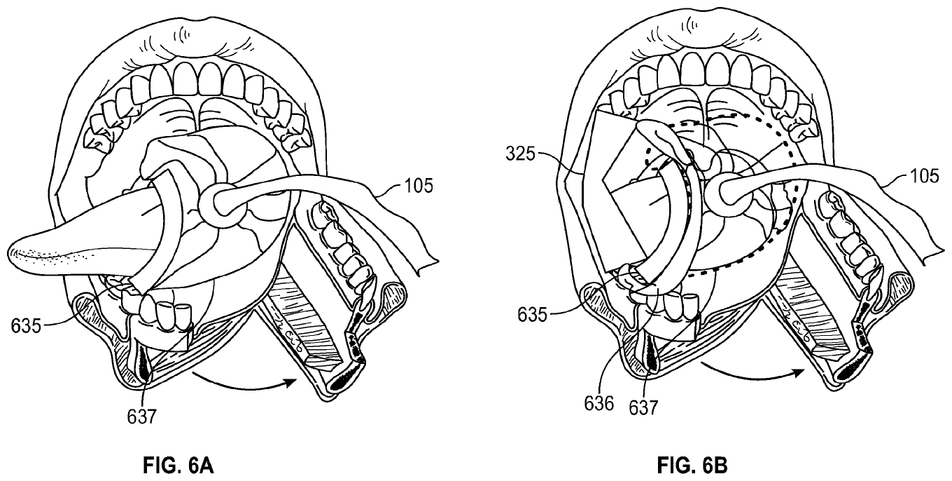

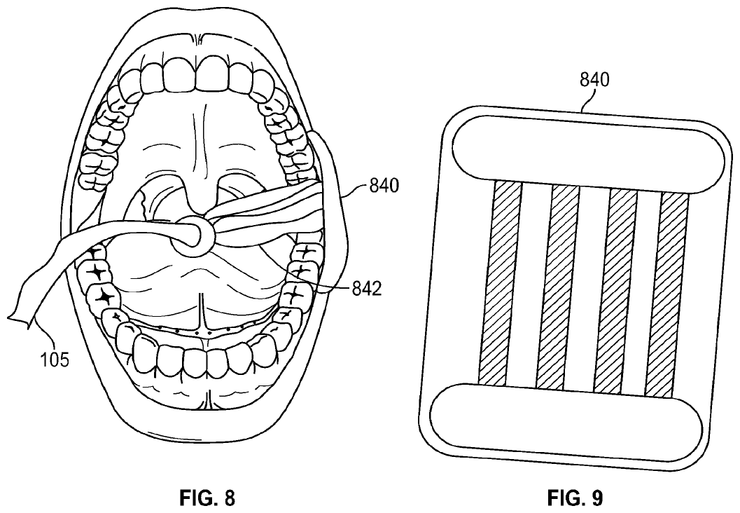

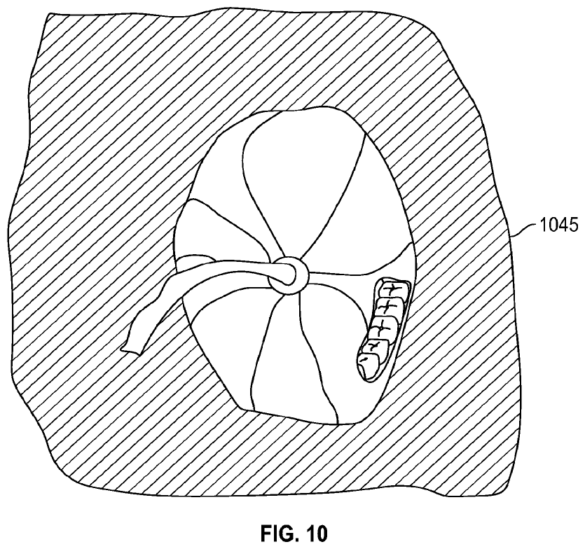

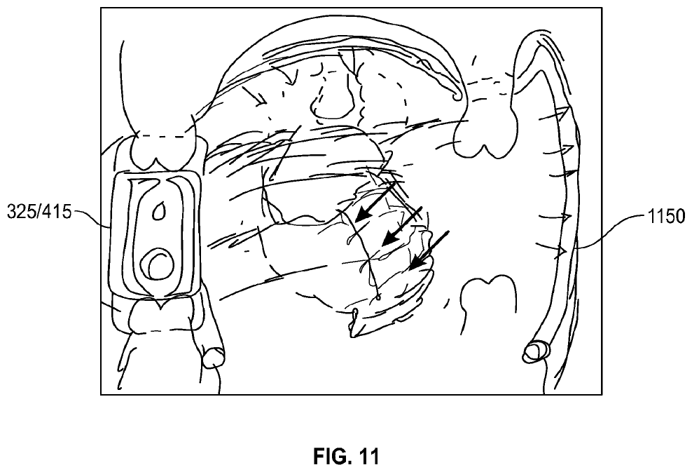

Membrane 110 can be inflated (110b) or deflated (110a) in accordance with current inputs. Membrane 110 can include separately inflatable and vacuum chambers, pockets and tubes, integrated inflatable support structures, and channels with holes for the removal of saliva and debris that can be separately controlled based on a given input. As will be discussed below, membrane 110 may include and/or be integrated with one or more access ports (FIG. 2B), bite blocks (e.g., FIGS. 3A-4), tongue retractors (e.g., FIGS. 6A-6B), and/or cheek retractors (e.g., FIGS. 8-9). By controlling compressed air and/or vacuums provided to various channels of the membrane 110, the access ports, bite blocks, tongue retractors, and/or cheek retractors may be operated as needed or desired. Membrane 110 may be flavored and/or include a disposable flavored covering.

In some cases, III 100 may include heating and/or cooling elements. For example, III 100 may include a conductive membrane to transfer heat and/or coolness to individual parts of the mouth (e.g., teeth, gums, tongue, etc.) as needed for comfort or procedures. In certain cases, III 100 may provide massage and/or tissue stimulation to individual parts of the mouth (e.g., gums, tongue). Massage and/or tissue stimulation can be provided by alternating compression of specific areas. For example, the membrane 110 may include a compression pocket or sleeve surrounding a specific area; by alternatingly applying compressed air to the pocket or sleeve, the specific area may be massaged. However, this is merely an example. In some cases, III 100 could include a vacuum cup. Negative vacuum pressure could be applied to provide “cupping therapy” and/or pulsed to provide a massage. In some cases, electrical stimulation therapy may be provided through electrodes disposed within or attached to membrane 110. In some cases, a water channel may be provided through or around membrane 110, and water pulsation massage may be provide. The water from the water pulsation massage may then be retrieved through a vacuum tube/channel

In some cases, III 100 may include an rapid emergency deflation mechanism. For example, membrane 110 could include and emergency deflation port that can quickly open and deflate the membrane 110 for emergency removal. As an example, membrane 110 may include a stitched seam connected to a pull cord. When pulled, the seam would rip, rapidly deflating membrane 110.

FIGS. 2A and 2B illustrate an inflatable intraoral isolation device (III) 100 in inflated and deflated states within a patient's mouth. As seen in FIG. 2A a deflated III 100 is inserted into the patient's mouth. Then, compressed air is provided via umbilical 105, and the III inflates (FIG. B). An access port 220 is provided so that a lower left section of teeth may be accessed. However, a remainder of the patient's mouth is protected and covered. Although port 220 reveals a lower left section of teeth, this is merely an example. One of ordinary skill would recognize that access port 220 may be positioned to reveal different sections of teeth. Additionally, III 100 could include a plurality of access ports 220 that provide access to different sections of teeth. By applying compressed air and/or vacuum to different channels of membrane 110, different access ports 220 may be hidden and revealed.

By inflating to within a patient's mouth, a patient's throat can be isolated to a high degree and, as described below in greater detail, breathing and/or inhalation management of a patient is possible. The membrane 110 protects the throat from broken instruments, dropped instruments, broken tooth segments and all other debris that might otherwise fall into the patients' throat. Additionally, III 100 may provide some noise abatement (e.g., by blocking and/or absorbing noise from a procedure). Additionally, in some cases, III 100 can include active noise reduction technology (e.g., negative wave technology) for significant noise abatement. In some cases, III 100 can provide music to a patient during the procedure.

When inflated, membrane 110 may be dimensioned substantially similar to an oral cavity to substantially fill a patient's mouth. However, this is merely an example, and, in some cases, membrane 110 can form various shapes, for example, a cup shape, a ramp shape (e.g., ramping away from access port 220), and or various other configurations. In this way, a space for an operation may be increased while continuing to provide isolation and protection.

III 100 can be dynamically inflated within a patient's mouth in order to adjust for a correct sizing. For example, a particular amount of pressure may be used to inflate III 100 to a certain size based on an amount of resistance from a patient's hard and soft tissue. Increasing the pressure can force a patient's mouth to open further (e.g., providing inflation to the bite block can increase the distance between teeth opening the jaw). Additionally, by selectively increasing the pressure to specific regions, a patient's oral cavity can be manipulated to provide improved access for a dental procedure (e.g., providing inflation can increase the retraction of soft tissues such as the patient's tongue and/or cheek).

Additionally or alternatively, scans of a patient (e.g., scanned models of a specific patients' maxilla and mandible or directly scanned patient arches) may be used to determine inflation characteristics for a particular patient. Further, the nature of the procedure planned may be considered in the design and/or inflation of the III 100. For example, if the dentist plans a crown procedure on a lower right molar, the III 100 may include a bite block 325 on the left side, the access port 220 planned for that molar tooth and the two adjacent teeth, tongue 635 and cheek retraction 840 on the right side, intraoral light source, intraoral vacuum ports both lower right and lower left, and an attachment for a micro camera to be placed in the upper right area with a view of the tooth to be manipulated.

FIGS. 3A and 3B illustrate a bite block 325 according to some embodiments. Bite block 325 includes a bite surface 327 and an inflatable bladder 329. The bite surface 327 may include two parts separated by a hinge 328. By inflating the inflatable bladder 329, bite surface 327 may be forced to separate, thereby forcing open a patient's jaw in the sagittal plane as shown in FIG. 3B. In some cases, one or more inflatable stops 330 may be attached to bite block 325. The inflatable stops 330 may be configured to inflate behind distal teeth of a patient, thereby locking the bite block (and, if integrated, the III) in place. In some embodiments the bite block 325 may be located on the internal aspect of the membrane with the potential to retract the adjacent cheek as well.

FIG. 4 is an view of an III with a bite block 325 according to some embodiments. In FIG. 4, umbilical 405 leads to bite block 325. Accordingly, bite block 325 may act as a side port 415 in addition to a bite block 325. In such cases, III may not include a central port 115, but rather route all umbilicals through the bite block 325/415. Although bite block 325 is shown integrated into III, this is merely an example. In some cases, bite block 325 may be a standalone unit. Similarly, in some cases, stops 330 may be provided to III in the absent of bite block 325 and/or bite block 325 may be provided with stops 330. Although stops 330 may be disposed behind a distal tooth, this is merely an example. In some cases, a missing tooth space or any anatomical undercut may be utilized by an inflatable stop 330 placed to resist displacement of the III 100.

One of ordinary skill will recognize in light of the present disclosure that bite block 325 is not necessarily inflatable. In some cases, bite block 325 may be rigid and attached to membrane 110. As would be understood by one of ordinary skill, bite block 325 may generally be disposed on a side of a patient's mouth where work is not being conducted. However, there are circumstances where bite block 325 may be positioned on a same side of a patient's mouth where work is to be conducted and/or a bite block 635 may be positioned on each side of the patient's mouth. Furthermore, one or ordinary skill will recognize that bite block 325 may be disposed on either side of membrane 110 (e.g., either external or partially or completely internal to III 100).

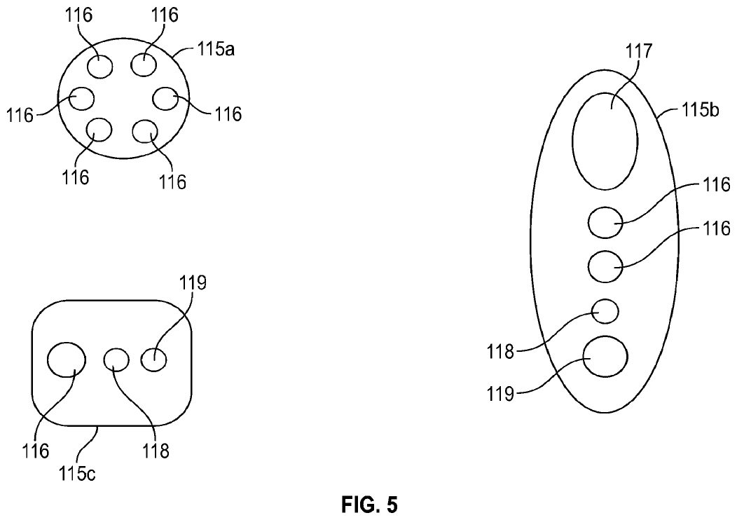

FIG. 5 illustrates example port layouts (e.g., port 115 or 415) in accordance with some embodiments. Port 115a includes six channels 116 distributed radially. Channels 116 may variously receive compressed air, vacuum, light, and/or power. In some cases, a plurality of channels 116 may receive compressed air to be routed to different portions of membrane 110. For example, a first channel 116 may route air to a primary membrane to control primary inflation and deflation, a second channel 116 may route air to access port 220 to control access thereto, and a third channel 116 may route air to inflatable bladder 329 of bite block 325 to control a size of opening. Accordingly, different amounts of compressed air (e.g., different pressures) may be provided to different portions of III 100. The compressed air routes may also be used simultaneously for negative wave sound dampening technology.

Port 115b includes two compressed air channels 116, a snorkel 117, a light channel 118, and vacuum channel 119. Port 115c includes a single compressed air channel 116, a light channel 118, and a vacuum channel 119. Light channel 118 may guide light to various portions of membrane 110, for example, to light a work area of a patient's mouth. The light may be used for visibility, disinfection, and/or treatment purposes. Additionally, by routing light through III 100 within a patient's mouth, visibility may be increased. In some cases, diffuse light may be provided through membrane 110. In this way, it limits blockage of the light from an operator's hands or instruments.

Vacuum channel 119 may provide vacuum force to control different aspects of III and/or to provide rapid deflation of III. One of ordinary skill will recognize that these channel provisions and configurations are for example purposes only, and one or more ports having one or more channels of the same and/or different type are anticipated within the scope of the present disclosure.

Snorkel 117 may provide, for example, oxygen, nitrous oxide, anesthesia gases and/or other inhalation agents. Snorkel 117 may provide a breathing pathway to a patient when III is in use. Snorkel 117 may contain a plurality of pathways. For example, snorkel 117 may contain four separate pathways, room air inhalation pathway, room air exhalation pathway, therapeutic gases inhalation pathway, and therapeutic gases exhalation pathway. One advantage of separate inhalation and exhalation routes is the ability to manage exhaust gases such as nitrous oxide such as venting to the external environment to protect the operators. In some cases, a nose mask may be provided instead of or in addition to snorkel 117. Accordingly, a patient would be able to more easily breathe through their nose and/or mouth depending on configuration.

FIGS. 6A and 6B illustrate example tongue retractor 635 in accordance with some embodiments. In FIG. 6A tongue retractor 635 is pushed from a central portion of membrane 100 by expander 637. When compressed air is provided to expander 637, tongue retractor 635 pushes a patient's tongue away from an access area. In another embodiment there may be an inflatable pocket, or armature located at the base of the tongue on the side intended for retraction. When this pocket is inflated (for example to 1.5 inches), retraction of the tongue is achieved. Varying levels of inflation can increase or decrease the amount of retraction. In FIG. 6B, tongue retractor 635 is actuated by vacuum piston 636, which pulls tongue retractor 635 towards bite block 325. In this way, either push, pull, or both may be used to control tongue retractor 635.

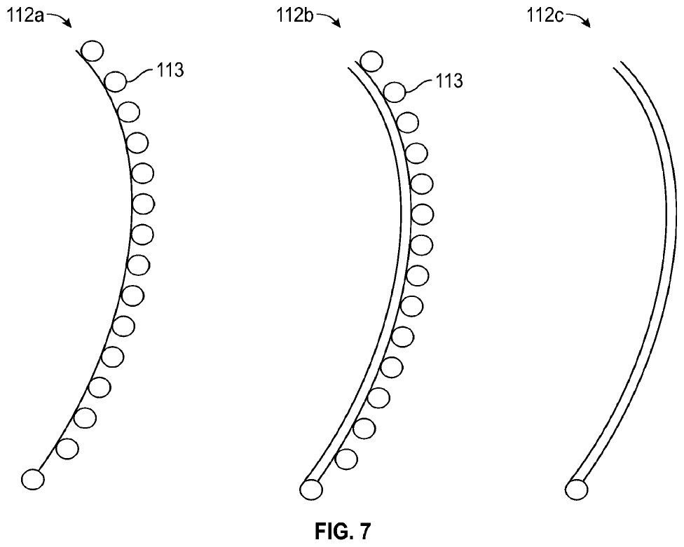

FIG. 7 illustrates example wall structures 112 of membrane 110 in accordance with some embodiments. Membrane 112a is single-walled and include a plurality of ribs to provide structural support, shape, and expansive force when inflated. Wall 112b is double walled and includes a plurality of ribs 113 to provide structural support, shape, and expansive force when inflated. Wall 112c is double walled but does not include any ribs. The membranes can include large area inflatable pockets or chambers. The membranes can also have more complex architectures of inflatability between layers of membranes. Double-walled membranes 110 may have a space between the walls (e.g., due to inflation) or the walls may be touching. In some cases, inflatable ribs, vacuum tubes, vacuum pistons, compressed air tubes, water tubes, adjunct communication pathways, and/or various types of light diffusion and distribution pathways may be included between the walls of membrane 110. One of ordinary skill will recognize that these are merely examples, and membrane 110 may have various numbers of walls and wall structures, including different structures at different portions within membrane 110.

FIG. 8 illustrates example a cheek retractor 840 in accordance with some embodiments. Cheek retractor 840 is pushed from a central portion of membrane 110 by expander 842. When compressed air is provided to expander 842, cheek retractor 840 pushes a patient's cheek outward, away from an access area. Cheek retractor 840 can include a cheek pad, gum pads, and inflatable ribs, as well as inflatable features for retraction and/or otherwise manipulation of any and all of the soft tissues of the buccal corridor.

In some cases, III 110 may include both tongue retractor 635 and cheek retractor 840. In such cases, a same air pressure may be applied to both expanders 637 and 842. However, this is merely an example. In some configurations, two different sources of air pressure may be applied, respectively, to the expander 637 and expander 842 for differential retraction of the different structures.

FIG. 9 illustrates an example internal structure of membrane 110, expander 637, and/or expander 842. The internal structure may include one or more inflatable ribs structural components to include inflatable resistance to displacement and compression thereof.

FIG. 10 illustrates an III with a drape 1045 in accordance with some embodiments. The drape may be provided at an outer edge of the III and/or an inner edge of the extraoral aspect (EVNES) to cover the patients' face, head, neck, and/or upper body. Drape 1045 may be made of fluid resistant and/or fluid absorptive materials such as a patient napkin with fluid resorptive away from the patients' body and fluid resistant toward their body. Drape 1045 may include any number of connectors for monitoring systems such as vital signs data, therapeutic adjunct systems such as provide radiation shielding with a window coincident with work to be done in which it may be advantageous to create radiographs throughout a procedure, entertainment systems, and comfort systems such as warming or cooling.

FIG. 11 illustrates an integrated vacuum 1150 in accordance with some embodiments. In FIG. 11, the vacuum 1150 is connected to bite block 325 as a side port 415. However, this is merely an example and, in some cases, the vacuum may be integrated with a central port 115 or membrane 110. The vacuum 1150 may provide for removal of saliva, liquid and/or debris during a procedure to keep a work area clear.

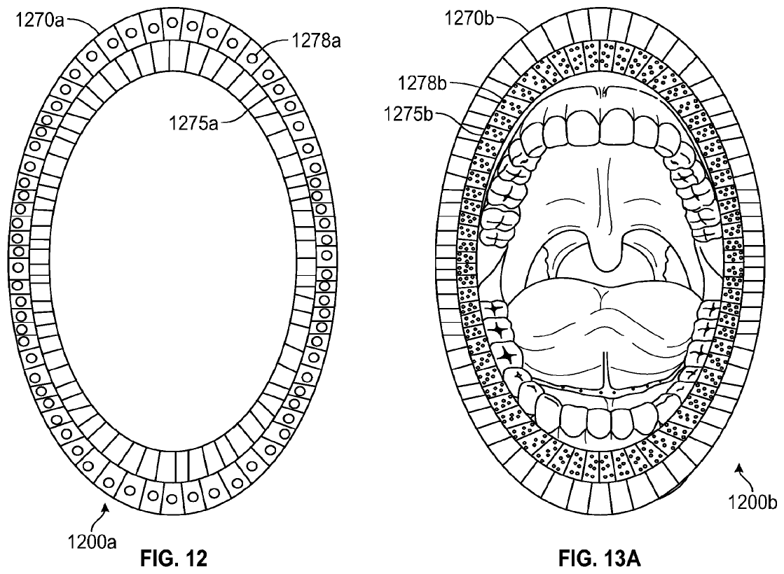

FIGS. 12-14 illustrate example extraoral vacuum negative environment systems (EVNES) 1200 in accordance with some embodiments. The EVNES 1200 may be circular, conical, or elliptical (e.g., mimicking a shape of the patients' mouth) or be of various other shapes that may enhance air dynamics for effective extraoral vacuum and negative environment production. The EVNES may be of various heights and/or variable heights. The EVNES may be constructed in a single layer or several layers and may be of varying thicknesses. The EVNES 1200 may provide light for visibility, light for disinfection, and extraoral vacuum and/or compressed air to create a negative air pressure environment that is at least partially contained by EVNES 1200. EVNES 1200 may include a drape 1045. EVNES 1200 may filter or otherwise disinfect air vacuumed from the patient.

The compressed air network and/or vacuum network could utilize a simple distribution system and use, for example, a simple rheostat for inflation, deflation, and compressed air distribution to EVNES 1200. The compressed air network and/or vacuum network could be more complex using a more complicated air manifold system and more than one rheostat. Further, the compressed air network and/or vacuum network could use other control systems such as complex manifolds using computer or microprocessor control. An example of sophisticated control and manipulation of EVNES 1200, a microprocessor could control both air pressure and vacuum throughout the system. An operator could manual instruct the EVNES 1200 to create various negative environment architectures and/or adjust EVNES 1200 architecture to address various splatter, microdroplet/aerosol situations. These shape capabilities could be further combined with the computerized control of compressed air and vacuum in and around the EVNES 1200 for highly sophisticated functionality design possibilities.

Referring to FIG. 12, EVNES 1200a includes an upper portion 1270a and a wall 1275. The upper portion 1270 includes a plurality of ports 1278 (e.g., vacuum ports and/or compressed air ports) disposed thereon. A vacuum may be provided from ports 1278 in order to reduce the amount of spray, aerosol, and/or debris exiting a patient's mouth. In some cases, EVNES 1200 may provide an isolated or substantially isolated environment for operating on a patient's mouth Wall 1275 may be a substantially vertical or angled wall extending from a patient's mouth. Portions of wall 1275 may be rigid, semi-rigid, or flexible, and may be made, for example, out of nylon, plastic, and/or silicon. In some cases, wall 1275 may be inflatable (e.g., FIG. 16A). In certain cases, wall 1275 may have an accordion structure (e.g., FIG. 16B) and/or may be formed by a compressible/springy material or structure. In some cases, wall 1275 may taper (e.g., be narrower at a top portion).

Wall 1275 may be collapsible against the force of an operator or assistant's a hand(s), finger(s) and/or instrument(s) when placed into or around the patients' mouth. In the course of a dynamic procedure where hands and instruments are brought into and out of a patients' mouth, EVNES 1200 or sections thereof can be repeatedly collapsed when the hand or instrument is inserted and rebound to its shape through resilient materials and/or reinflation when the object is removed. Thus, the EVNES 1200 can provide an isolated environment without interfering with dental operations. EVNES 1200 can have an adaptable shape using one or more of vacuum tubes, ports, pockets, and pistons and compressed air tubes, ports, pockets, chambers, and ribs to enable a wide variety different architectures of (positive) and negative air pressure environments close to the patients mouth.

In some cases, an operator may utilize one or more replacement shields (e.g., on the back of the operator's hand) This replacement shield may improve suctioning effects/splatter reduction when the operator moves the wall if the EVNES (e.g., during operation). The shield may contribute to containing debris, aerosols, et cetera, while also reducing vacuum loss within the negative environment system from compression of the wall.

There may also be a shield above the wall(s) of the EVNES. These shields may perch above and be rigid (or semi-rigid), such that they do not greatly adapt to pressure from tools and an operator hands. These shields may be used to further define the boundaries of the negative environment and improve negative pressure zone.

In some implementations, the EVNES wall may be configured to surround an object (e.g., the operator's hand or a tool) that compressed the wall. Thus, the walls may allow easy access to the interior while helping to maintain the negative environment (e.g., because the structure minimizes gaps during compression).

Wall 1275 may be less that 1 mm thick or may be up to 3 centimeters thick and may contain several layers or ply's which may in turn contain different types of functionality within the different layers. These layers may be constructed in a way that allows further shape possibilities of the negative environment system with use of rigid materials like plastic, vacuum collapsible pistons, and/or compressed air pistons or areas of inflatability. The EVNES 1200 could be variably inflatable with different layers of variable inflatability and/or different chambers of variable inflatability and collapsibility.

In some embodiments, the air may be used to aid in visibility, such as by providing defogging characteristics or addressing breathing and other concerns of negative and positive pressure environments.

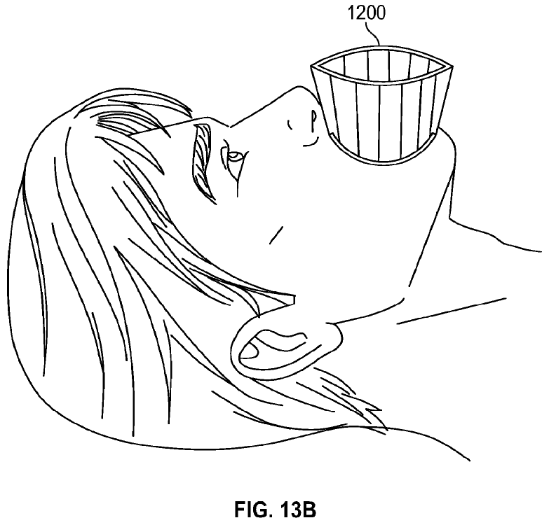

Referring to FIGS. 13A and 13B, EVNES 1200b has been placed around a patient's mouth, at approximately the orifice or the patient lips. By placing EVNES 1200 around a patient's mouth, it can effectively follow a patient as they move. EVNES 1200b includes vacuum ports 1278 on wall 1275. As in FIG. 12, the vacuum ports may provide a vacuuming to remove spray, aerosol, microdroplets, and/or debris exiting a patient's mouth. The ports 1278 located on wall 1275 may be in addition to or instead of ports disposed on upper portion 1270. One of ordinary skill in light of the present disclosure will recognize that these ports may be of varying size and shape and may have different orientations in regard to the membrane.

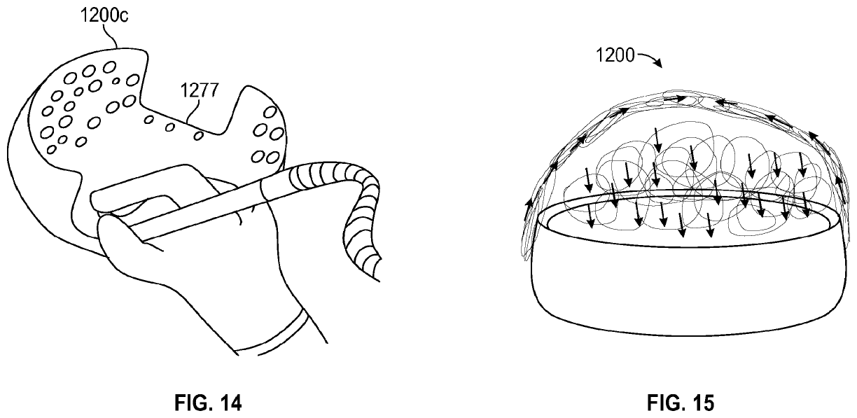

In some cases, as shown in FIG. 14, EVNES 1200c may include cut-outs 1277 for operator hands, fingers, and/or instruments. In this case, the EVNES 1200c may be more rigid, and the need to collapse is reduced. The cutouts 1277 may be on one or both sides when disposed on a patient's mouth. In some cases, a single cutout 1277 may be provided. The EVNES 1200 may be adjustable and rotatable such the cutout(s) 1277 may be positioned as needed for a given operation.

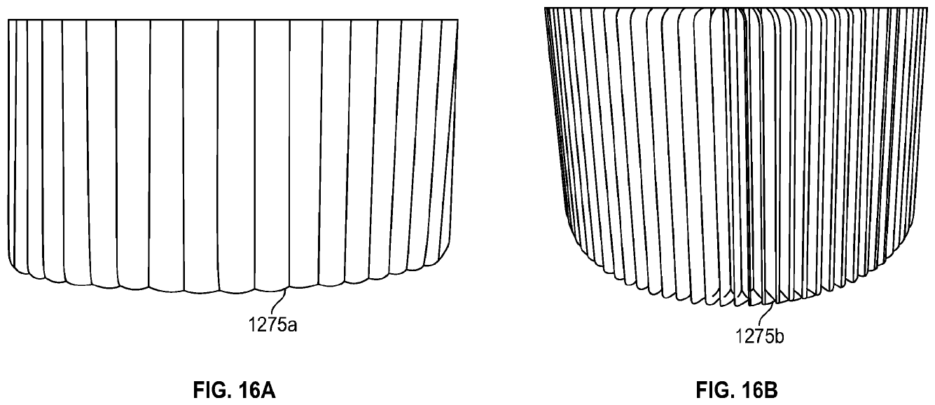

FIG. 15 illustrates a negative environment created by EVNES 1200. In other embodiments, various air outlets 1278 that may be located on upper portion 1270a and/or on an outer surface of wall 1275 push out compressed air utilizing higher pressure on an outer aspect (e.g., 1270) to create positive air pressure around the outer aspects of the EVNES 1200, while vacuum ports 1278 utilize vacuum in the rim and inner aspects (e.g., wall 1275) to create negative air pressure. The cone of high pressure and low pressure can create a negative environment for amelioration or elimination of splatter, microdroplets and aerosols from the patient's mouth.

EVNES may be secured in a variety of ways. For example, the EVNES may be secured to the patient using strap(s), clip(s), an adhesive, staple(s), and/or suture(s). Additionally, the EVNES may be applied as part of a larger system to include a shield above the EVNES to intercept any splatter but in some cases to also contribute to the confines of the negative environment system. In some cases, the EVNES may have various intra-oral platforms to secure the EVNES to a patient. For example, EVNES may include an isolite or isolite type platform, which can provide intraoral vacuum, light (e.g., for sterilization and/or visibility), a bite block, and tissue retraction, or could be include a rubber dam type platform oriented in a way that the patient's breath will be captured by the EVNES. The EVNES may include a camera or a mounting point for camera equipment.

In some cases, the EVNES may include barbs or other means of securing the rubber dam. For example, barbs or attachment points may be placed on the EVNES frame in a way the patient may breathe, e.g., through an oral cavity between the rubber dam and the patients lips. Further, in some cases, the attachment points may position the combination such that all phases of patient breathing occurs inside of the negative environment system. An EVNES drape may be located outboard of all rubber dam material so that no patient breath may escape the negative environment.

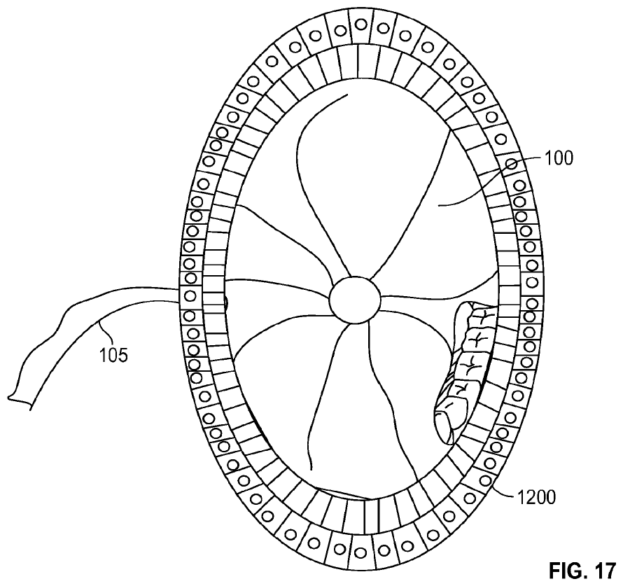

FIG. 17 illustrates an integrated III 100 an EVNES 1200 according to some embodiments. III 100 may secure EVNES 1200 to a desired location. In some cases, umbilical 105 may provide vacuum (e.g., to ports 1278) and/or compressed air (e.g., to wall 1275) and/or light to EVNES 1200.

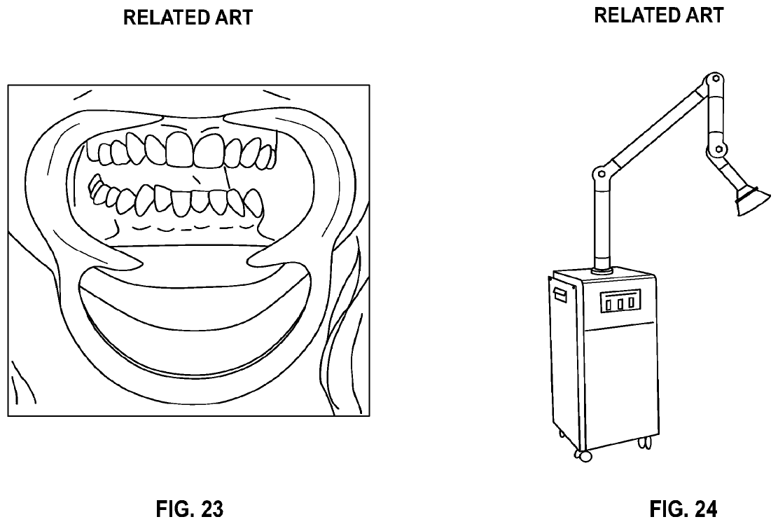

Although III 100 is illustrated as providing a securing mechanism to EVNES 1200, this is merely an example. In some cases, a strap may hold EVNES 1200 in a an appropriate position on a patient. One of ordinary skill would recognize in light of the present disclosure that EVNES 1200 may be connected to or integrated with an alternative device, such as a bite block or a cheek retractor (e.g., FIG. 23), as would be known by one of ordinary skill.

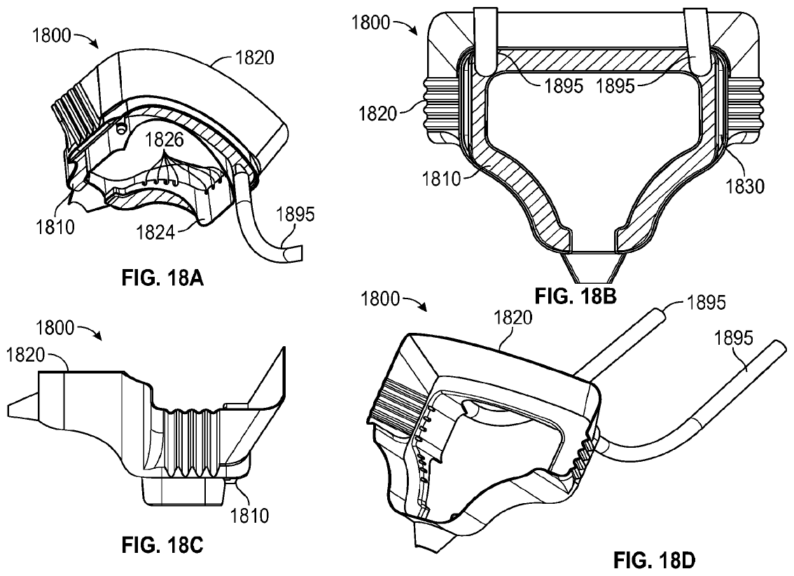

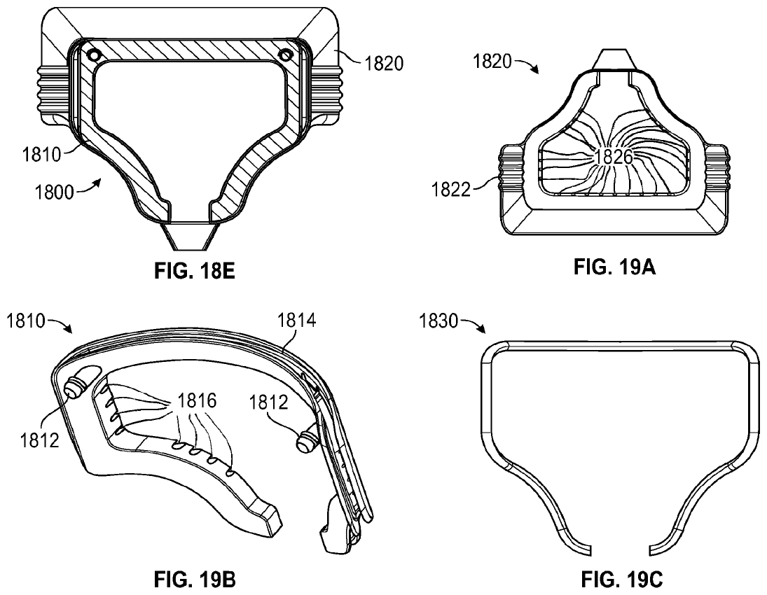

FIGS. 18A-E and FIGS. 19A-19C illustrate an EVNES 1800 according to an example embodiment. The EVNES 1800 includes a frame 1810, a hood 1820, and a seal 1830. The frame 1810 may provide rigid or semi-rigid support to the EVNES and may be made out of a hard plastic. The frame 1810 may include one or more air ports 1812, an seal insert 1814, and one or more air pores 1816. The air ports 1812 may receive air and/or vacuum supply tubes (1895) from which positive airflow and/or vacuum pressure may be supplied to the EVNES 1800. Sean insert 1814 may provide a channel for the seal 1830. Air pores 1616 may provide an exit path for the air and/or vacuum into a central portion of the EVNES 1800. The frame 1810 may be shaped to substantially conform to a patient's mouth (or other portion of the patient's body). As discussed, the frame 1810 may be designed to generally fit an oral or surgical location, or may be specifically designed and formed for the patient.

Hood 1820 may be affixed to frame 1810. Hood 1820 may include a wall 1822, a drape 1824, and air pores 1826. Wall 1822 may be substantially similar to wall 1275 discussed elsewhere. For example, wall 1822 may surround the negative environment and provide an entry point for an operator's hands and/or tools. The wall 1822 may include an accordion portion designed to more deform in respond to pressure from an operator. Drape 1824 may be on an interior of frame 1810, and may provide a substantial seal between the EVNES 1800 and the patient. Air pores 1826 may be provided to allow air and/or vacuum to enter the negative environment. The air pores 1826 may substantially align with the air pores 1816 of the frame 1810. However, this is merely an example. IN some cases, air channels and/or other structures may be provided within hood 1820 to guide the air and/or vacuum to specific points within the negative environment. Hood 1820 may be made of, for example silicon. In some cases, hood 1820 may be disposable. Hood 1820 may be formed for a specific patient and/or a potential use. Seal 1830 provides a seal between frame 1810 and hood 1820, and helps to secure the EVNES 1800.

In some cases, the use of an III or an EVNES may make breathing more difficult. Accordingly, in some cases, therapeutic or other gases may be introduced into the oral or nasal cavity of the patient to ease breathing. A tube or port for providing air to the patient may be integrated into the III or the EVNES. Breathing support may be provided by intubation, positive pressure intraorally, and/or a nasal route (e.g., a nasal cannula with positive pressure air or therapeutic gases).

In some cases, the EVNES may include a drape of sufficient length to accommodate movement around the patient, for example, as the EVNES and/or patient is manipulate by the operator. Accordingly, the drape may still provide a substantially isolated environment.

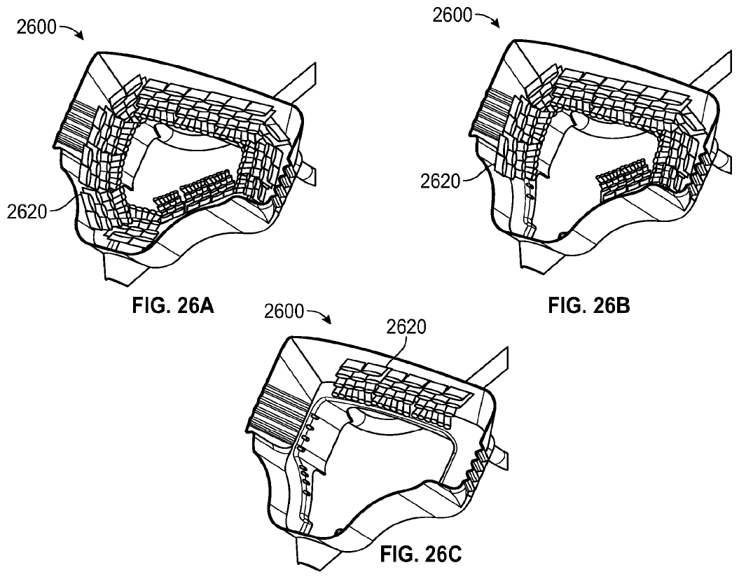

FIGS. 26A-26C illustrates an EVNES 2600 according to some embodiments. EVNES 2600 may be substantially similar to EVNES 1200 and 1800 described in relationship to the other figures. However, EVNES 2600 may include a plurality of layers vacuum tube ports 2620 formed along the wall 2610. The layers of ports 2620 may be generally positioned to face a focal site of the negative environment (e.g., the patient's mouth or surgery operation) (e.g., FIG. 26A). In some cases, the ports 2620 could be organized to create a vortex-like air current within the negative environment when the vacuum is applied. The stacks of ports 2620 may form a slanted, curved, or parabolic shape. A size and/or shape of the ports 2620 may differ. In some cases, the ports 2620 may extend from a base of the EVNES 2620 (e.g., a frame) and terminate at different heights. However, this is merely an example and, in some cases, ports 2620 may extend from the wall and terminate at different lengths. In some cases, the layers of ports 2620 may not extend around the entirety of the EVNES 2620 (e.g., FIG. 26B). For example, in some instances, the layers of ports 2620 may not extend over a nose of the patient (i.e., when the EVNES is in use). Likewise, in some cases, the layers of ports 2620 may be limited to specific areas around the EVNES (e.g., FIG. 26C). For example, the layers of ports 2620 may be provided along a bottom side (e.g., for working on upper teeth), a left-side (e.g., for working on the right side of a patient), or a right side (e.g., for working on a left side of a patient. The ports 2620 may be formed of a soft or semi-rigid material, or have various material qualities in various portions. For example, ports 2620 closer to the patient may be software and more deformable than ports farther away from the patient, but this is merely an example.

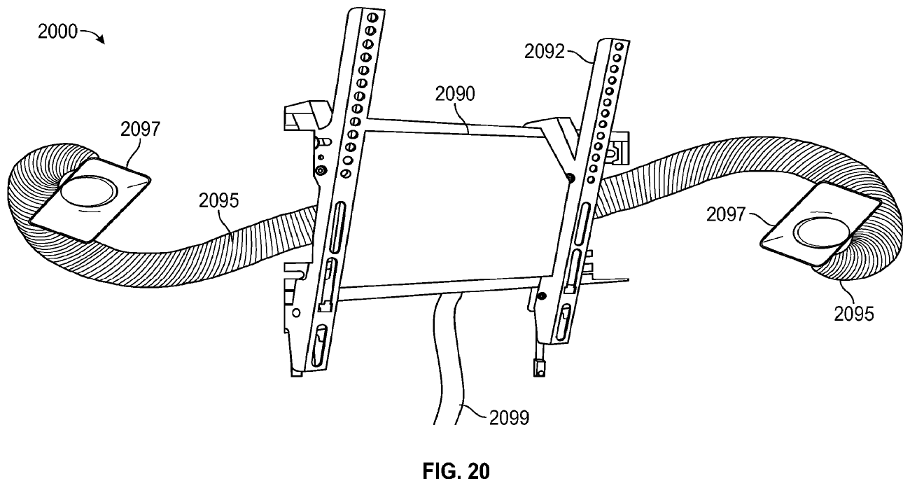

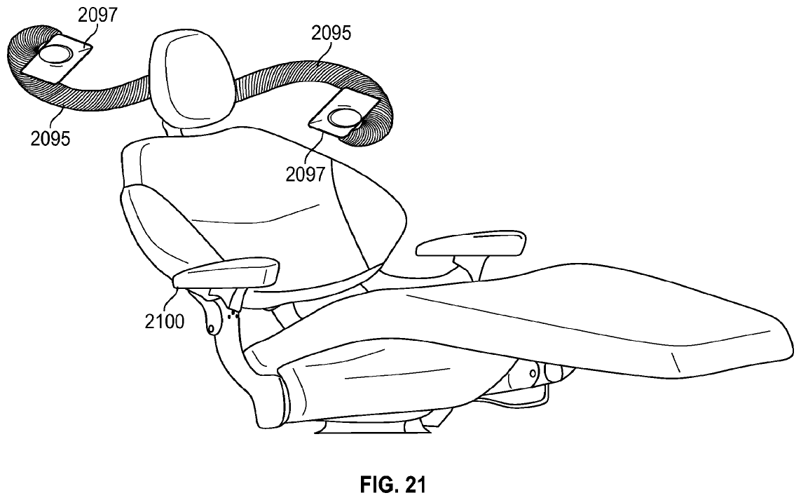

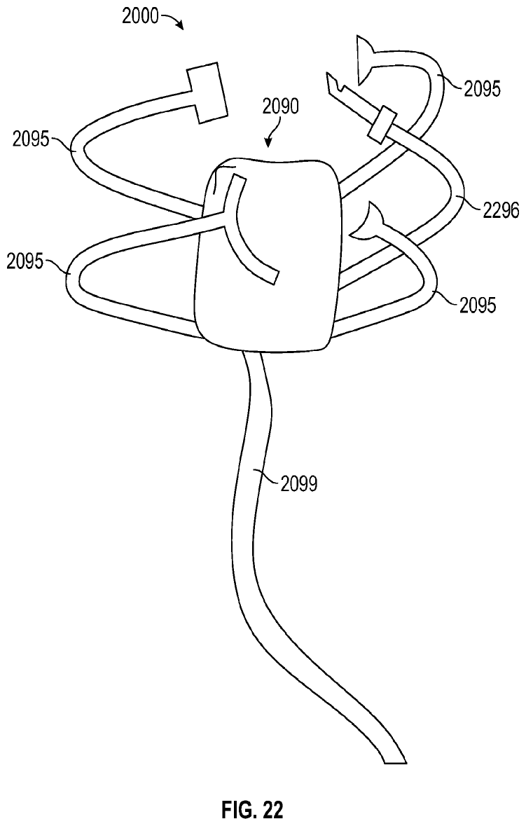

FIGS. 20-22 illustrate an example headrest negative environment systems (HNES) 2000 according to some embodiments. HNES 2000 can include a base 2090 and one or more vacuum arms 2095. Base 2090 may include one or more connection brackets 2092 or another attachment mechanism to attach HNES 2000 to a dental chair 2100. Arms 2185 may have a vacuum port 2097 disposed thereon. Arms 2095 may be articulating and semi-rigid such that arms 2095 can be manipulated to position ports 2097 to remove spray, aerosol, microdroplets, and/or debris exiting a patient's mouth. Umbilical 2099 can connect HNES 2000 to a vacuum source to power the ports 2097. In some cases, HNES 2000 may include one or two or more articulating vacuum arms 2095. In some embodiments, HNES 2000 may further include articulating arms to provide an additional function, such as articulating arm 2296 that provides light to a dental area.

As discussed, in some cases, the EVNES may provide a negative environment around a surgical site on the body. The EVNES (or the hood thereof) may be custom created (e.g., with a 3D printer or cut to size) based on a topography of the surgical site. For example, the surgical site may be analyzed by scanning, photography, radar, lidar, or any other mean, and a shape of the EVNES and/or hood may be determined. In some cases, the EVNES may be partially embedded within a surgical site to provide greater isolation to the surgical site from surrounding tissue.

The EVNES may have any a plurality of ports (e.g., 1278) with a combination of sizes and/shapes. Within the EVNES, there may be positive pressure air to aid in visibility (e.g., defogging), addressing breathing, and/or providing additional features.

Additionally, as would be understood by one of ordinary skill, similar approaches could be used to provide a local positive pressure environment. For example, the use of a local positive pressure environment may provide gases for therapy, infection control, sterilization, pasteurization, and/or local hyperbaric treatment.

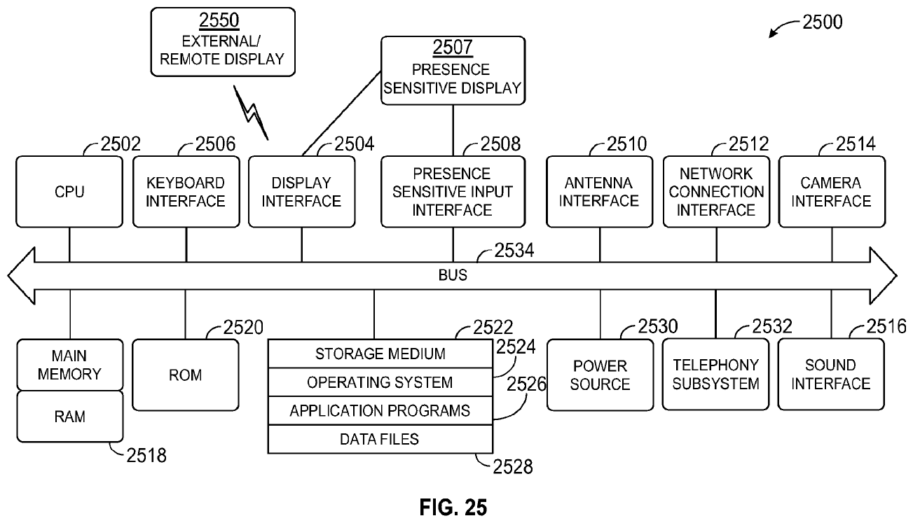

FIG. 25 is a block diagram of an illustrative computer system architecture 2500, according to an example implementation. As non-limiting examples, processors, microprocessors, and/or controllers of III 250, EVNES 1200, and/or EVNES 2000 may be implemented using one or more elements from the computer system architecture 2500. It will be understood that the computing device architecture 2500 is provided for example purposes only and does not limit the scope of the various implementations of the present disclosed systems, methods, and computer-readable mediums.

The computing device architecture 2500 of FIG. 25 includes a central processing unit (CPU) 2502, where computer instructions are processed, and a display interface 2504 that acts as a communication interface and provides functions for rendering video, graphics, images, and texts on the display. In certain example implementations of the disclosed technology, the display interface 2504 may be directly connected to a local display, such as a touch-screen display associated with a mobile computing device. In another example implementation, the display interface 2504 may be configured for providing data, images, and other information for an external/remote display 2550 that is not necessarily physically connected to the mobile computing device. For example, a desktop monitor may be used for mirroring graphics and other information that is presented on a mobile computing device. In certain example implementations, the display interface 2504 may wirelessly communicate, for example, via a Wi-Fi channel or other available network connection interface 2512 to the external/remote display 2550.

In an example implementation, the network connection interface 2512 may be configured as a communication interface and may provide functions for rendering video, graphics, images, text, other information, or any combination thereof on the display. In one example, a communication interface may include a serial port, a parallel port, a general-purpose input and output (GPIO) port, a game port, a universal serial bus (USB), a micro-USB port, a high definition multimedia (HDMI) port, a video port, an audio port, a Bluetooth port, a near-field communication (NFC) port, another like communication interface, or any combination thereof. In one example, the display interface 2504 may be operatively coupled to a local display, such as a touch-screen display associated with a mobile device. In another example, the display interface 2504 may be configured to provide video, graphics, images, text, other information, or any combination thereof for an external/remote display 2550 that is not necessarily connected to the mobile computing device. In one example, a desktop monitor may be used for mirroring or extending graphical information that may be presented on a mobile device. In another example, the display interface 2504 may wirelessly communicate, for example, via the network connection interface 2512 such as a Wi-Fi transceiver to the external/remote display 2550.

The computing device architecture 2500 may include a keyboard interface 2506 that provides a communication interface to a keyboard. In one example implementation, the computing device architecture 2500 may include a presence-sensitive display interface 2508 for connecting to a presence-sensitive display 2507. According to certain example implementations of the disclosed technology, the presence-sensitive display interface 2508 may provide a communication interface to various devices such as a pointing device, a touch screen, a depth camera, etc. which may or may not be associated with a display.

The computing device architecture 2500 may be configured to use an input device via one or more of input/output interfaces (for example, the keyboard interface 2506, the display interface 2504, the presence sensitive display interface 2508, network connection interface 2512, camera interface 2514, sound interface 2516, etc.) to allow a user to capture information into the computing device architecture 2500. The input device may include a mouse, a trackball, a directional pad, a track pad, a touch-verified track pad, a presence-sensitive track pad, a presence-sensitive display, a scroll wheel, a digital camera, a digital video camera, a web camera, a microphone, a sensor, a smartcard, and the like. Additionally, the input device may be integrated with the computing device architecture 2500 or may be a separate device. For example, the input device may be an accelerometer, a magnetometer, a digital camera, a microphone, and an optical sensor.

Example implementations of the computing device architecture 2500 may include an antenna interface 2510 that provides a communication interface to an antenna; a network connection interface 2512 that provides a communication interface to a network. As mentioned above, the display interface 2504 may be in communication with the network connection interface 2512, for example, to provide information for display on a remote display that is not directly connected or attached to the system. In certain implementations, a camera interface 2514 is provided that acts as a communication interface and provides functions for capturing digital images from a camera. In certain implementations, a sound interface 2516 is provided as a communication interface for converting sound into electrical signals using a microphone and for converting electrical signals into sound using a speaker. According to example implementations, a random-access memory (RAM) 2518 is provided, where computer instructions and data may be stored in a volatile memory device for processing by the CPU 2502.

According to an example implementation, the computing device architecture 2500 includes a read-only memory (ROM) 2520 where invariant low-level system code or data for basic system functions such as basic input and output (I/O), startup, or reception of keystrokes from a keyboard are stored in a non-volatile memory device. According to an example implementation, the computing device architecture 2500 includes a storage medium 2522 or other suitable type of memory (e.g. such as RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives), where the files include an operating system 2524, application programs 2526 (including, for example, a web browser application, a widget or gadget engine, and or other applications, as necessary) and data files 2528 are stored. According to an example implementation, the computing device architecture 2500 includes a power source 2530 that provides an appropriate alternating current (AC) or direct current (DC) to power components.

According to an example implementation, the computing device architecture 2500 includes a telephony subsystem 2532 that allows the device 2500 to transmit and receive sound over a telephone network. The constituent devices and the CPU 2502 communicate with each other over a bus 2534.

According to an example implementation, the CPU 2502 has appropriate structure to be a computer processor. In one arrangement, the CPU 2502 may include more than one processing unit. The RAM 2518 interfaces with the computer bus 2534 to provide quick RAM storage to the CPU 2502 during the execution of software programs such as the operating system application programs, and device drivers. More specifically, the CPU 2502 loads computer-executable process steps from the storage medium 2522 or other media into a field of the RAM 2518 to execute software programs. Data may be stored in the RAM 2518, where the data may be accessed by the computer CPU 2502 during execution.

The storage medium 2522 itself may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DVD) optical disc drive, an internal hard disk drive, a Blu-Ray optical disc drive, or a Holographic Digital Data Storage (HDDS) optical disc drive, an external mini-dual in-line memory module (DIMM) synchronous dynamic random access memory (SDRAM), or an external micro-DIMM SDRAM. Such computer readable storage media allow a computing device to access computer-executable process steps, application programs and the like, stored on removable and non-removable memory media, to off-load data from the device or to upload data onto the device. A computer program product, such as one utilizing a communication system may be tangibly embodied in storage medium 2522, which may include a machine-readable storage medium.

According to one example implementation, the term computing device, as used herein, may be a CPU, or conceptualized as a CPU (for example, the CPU 2502 of FIG. 25). In this example implementation, the computing device (CPU) may be coupled, connected, and/or in communication with one or more peripheral devices, such as display. In another example implementation, the term computing device, as used herein, may refer to a mobile computing device such as a Smartphone, tablet computer, or smart watch. In this example implementation, the computing device may output content to its local display and/or speaker(s). In another example implementation, the computing device may output content to an external display device (e.g., over Wi-Fi) such as a TV or an external computing system.

In example implementations of the disclosed technology, a computing device may include any number of hardware and/or software applications that are executed to facilitate any of the operations. In example implementations, one or more I/O interfaces may facilitate communication between the computing device and one or more input/output devices. For example, a universal serial bus port, a serial port, a disk drive, a CD-ROM drive, and/or one or more user interface devices, such as a display, keyboard, keypad, mouse, control panel, touch screen display, microphone, etc., may facilitate user interaction with the computing device. The one or more I/O interfaces may be used to receive or collect data and/or user instructions from a wide variety of input devices. Received data may be processed by one or more computer processors as desired in various implementations of the disclosed technology and/or stored in one or more memory devices.

One or more network interfaces may facilitate connection of the computing device inputs and outputs to one or more suitable networks and/or connections; for example, the connections that facilitate communication with any number of sensors associated with the system. The one or more network interfaces may further facilitate connection to one or more suitable networks; for example, a local area network, a wide area network, the Internet, a cellular network, a radio frequency network, a Bluetooth enabled network, a Wi-Fi enabled network, a satellite-based network any wired network, any wireless network, etc., for communication with external devices and/or systems.

As used in this application, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

As an example, embodiments or implementations of the disclosed technology may provide for a computer program product, including a computer-usable medium having a computer-readable program code or program instructions embodied therein, said computer-readable program code adapted to be executed to implement one or more functions specified in the flow diagram block or blocks. Likewise, the computer program instructions may be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

In this description, numerous specific details have been set forth. It is to be understood, however, that implementations of the disclosed technology may be practiced without these specific details. In other instances, well-known methods, structures and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form. By “comprising” or “containing” or “including” is meant that at least the named element, or method step is present in article or method, but does not exclude the presence of other elements or method steps, even if the other such elements or method steps have the same function as what is named

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain embodiments of this disclosure have been described in connection with what is presently considered to be the most practical and various embodiments, it is to be understood that this disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the technology and also to enable any person skilled in the art to practice certain embodiments of this technology, including making and using any apparatuses or systems and performing any incorporated methods. The patentable scope of certain embodiments of the technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. An intraoral inflatable isolator (III) for an oral cavity of a patient, the III comprising:
a compressed-air port; and
an inflatable membrane dimensioned to be insertable into the mouth and connected to the compressed-air port, the membrane being configured to inflate upon application of compressed air from the compressed-air port to isolate within the oral cavity.

2. The III of claim 1, further comprising an inflatable bite block.

3. The III of claim 2, further comprising an inflatable bite block configured to provide adjustable opening of a mouth of the patient in the sagittal plane based on an inflation amount thereof.

4. The III of claim 1, wherein the membrane comprises a plurality of inflatable features configured to expand the isolator in specific directions based on inflation of the inflatable features.

5. The III of claim 1 further comprising a tongue retractor and expander.

6. The III of claim 1 further comprising a cheek retractor and expander.

7. The III of claim 1, wherein the membrane further comprises one or more controllable access ports configured to individually reveal separate portions of the oral cavity.

8. (canceled)

9. The III of claim 1, further comprising an active noise reduction system configured to reduce apparent noise of the patient.

10. The III of claim 1, further comprising:
a vacuum port; and
a vacuum channel configured to provide extraction within the oral cavity.

11. The III of claim 1 further comprising a snorkel configured to provide a breathing pathway to the patient.

12. The III of claim 11, wherein the snorkel comprises a first pathway for breathing air and a second pathway for introduction of inhalation drugs.

13. The III of claim 11, wherein the snorkel comprises a first pathway for breathing in air and a second pathway for breathing out air.

14. The III of claim 1 further comprising at least one of:
a plurality of integrated vacuum pistons to manipulate a shape of the membrane;
an umbilical connector configured to provide at least one of compressed air, vacuum, or light;
a rapid emergency deflation system; or
a temperature control element configured to heat or cool selective portions of the membrane.

15-16. (canceled)

17. The III of claim 14, wherein the rapid emergency deflation system comprises a vacuum port configured to rapidly withdraw air from the membrane.

18-20. (canceled)

21. The III of claim 1 further comprising at least one of a disposable bite block, a massage element, a drape, an inflatable stop, or an inflatable anchorage.

22-24. (canceled)

25. The III of claim 1 further comprising an extraoral vacuum negative environment system (EVNES).

26. An extraoral vacuum negative environment system (EVNES) comprising:
a wall sized to fit around an orifice of a patient; and
a plurality of vacuum ports disposed within the wall such that a vacuum applied from the plurality of vacuum ports generates a negative environment surround the EVNES.

27. The EVNES of claim 26 further comprising a plurality of air outlets disposed within the wall such that air expelled from the plurality of air outlets generates a positive air pressure space around the negative environment.

28. The EVNES of claim 26 further comprising a plurality of air outlets disposed within the wall such that air expelled from the plurality of air outlets generates an air curtain around the negative environment.

29. The EVNES of claim 26 further comprising a plurality of air outlets disposed within the wall exterior to the plurality of vacuum ports.

30. The EVNES of claim 26, wherein the wall comprises an inflatable membrane.

31. The EVNES of claim 26, wherein the wall comprises an accordion membrane.

32. The EVNES of claim 26, wherein the wall is collapsible and reboundable.

33. The EVNES of claim 26 further comprising one or more cut-outs disposed within the wall.

34-36. (canceled)

37. A system comprising:
an intraoral inflatable isolator (III) comprising:
a compressed-air port; and
an inflatable membrane dimensioned to be insertable into the mouth and connected to the compressed-air port, the membrane being configured to inflate upon application of compressed air from the compressed-air port to isolate within the oral cavity; and
an extraoral vacuum negative environment system (EVNES) comprising:
a wall sized to fit around an orifice of a patient; and
a plurality of vacuum ports disposed within the wall such that a vacuum applied from the plurality of vacuum ports generates a negative environment surround the EVNES.
